(19) United States
(12) Plant Patent Application Publication
(10) Pub. No.: US 2002/0092046 P1
(43) Pub. Date: Jul. 11, 2002

(54) Rose Plant named 'Tanmirsch'

(75) Inventor: Hans-Jurgen Evers, Uetersen (DE)

Correspondence Address: Russell D. Orkin, 700 Koppers Building, 436 Seventh Avenue, Pittsburgh, PA, 15219-1818, US

(73) Assignee: Rosen Tantau Mathias Tantau Nachfolger

(21) Appl. No.: 09/758,722

(22) Filed: Jan. 11, 2001

(51) Int. Cl. A01H 5/00

(52) U.S. Cl. PLT/104

(57) ABSTRACT

The new cultivar is a variety of bed rose plant having an bushy growth habit. The yellow flowers are stable in color. The plant is robust, exhibits good hardiness, and ground covering ability.

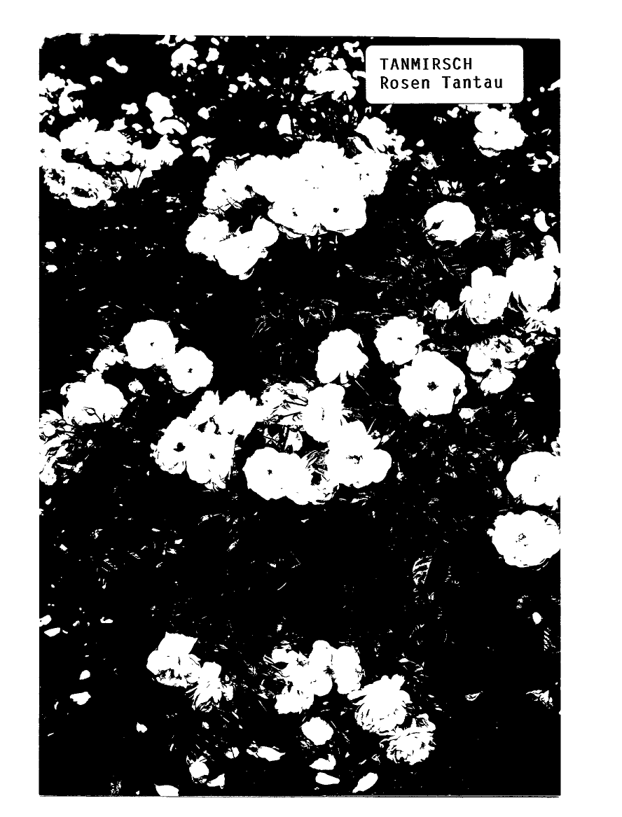

### BACKGROUND OF THE NEW PLANT

The present invention includes a new and distinct cultivar of Rosa l. known by the varietal name ‘Tanmirsch’.

The plant was discovered in 1988 by the breeder in Ueterson, Germany in a selective breeding program designed to produce a yellow bed rose variety that is robust, hardy, and has a stable color. The new plant is a selection from a cross of unnamed seedlings maintained proprietary by the assignee of the present application. The plant was first asexually reproduced in 1993 and can be propagated asexually on Rosa canina rootstock.

The variety is distinguishable by its very good hardiness, stable color, and good ground covering ability. Continued observations from the vegetative cuttings in Uetersen, Germany have confirmed that the distinguishing features of this new cultivar come true, remain stable, and are retained through successive propagations.

### DESCRIPTION OF THE DRAWING

The accompanying photographic drawing illustrates the new cultivar. Colors are described in part in reference to the R.H.S. Colour Chart of The Royal Horticultural Society of London.

### DESCRIPTION OF THE NEW PLANT

The following description sets forth the characteristics of the new cultivar.

The Plant:

Botanical name.—Rosa l.

Size:

Height.—60 cm.

Width.—50 cm.

Habit: Bushy.

Growth vigor: Vigorous.

Wood color: Green.

Thorns:

Color.—Brown-red.

Size: 0.5 cm.

Shape: Elongated.

Location on stems and branches: Random arrangement.

Disease resistance: No unusual susceptibility to diseases or pests has been noted to date.

Hardiness: Hardy.

### Foliage

Arrangement: Compound; unpaired pinnate.

Number of leaflets on typical stem leaves: 5.

Leaflets:

Size.—4.2 cm.

Color.—Green. Upper surface: Dark green. Lower surface: Bright green.

Shape.—Elliptical.

Texture.—Smooth.

Margin.—Serrated.

Petiole rachis:

Stipules.—Size: 0.9 cm. Shape: Elongated.

### Bud

Size: 1 cm.

Shape: Bowl-shaped.

Color: Yellow, becoming brighter during opening.

Sepals:

Color.—Green.

Texture.—Smooth.

Receptacle:

Color.—Greenish-white.

Shape.—Circular.

Texture.—Funnel-shaped.

### Flower

Borne (number in clusters): 3.

Size (diameter):

Cluster: 10 cm.

Blossom: 7 cm.

Form: Cup shaped.

Petals:

Number per blossom: 33.

Color.—Upper surface: Yellow Groups 4D-4B. Lower surface: Yellow Groups 4D-4B.

Texture.—Smooth.

Size.—3.8 cm.

Shape.—Elliptical.

Persistence.—High.

Lastingness.—High.

Fragrance: None.

Blooming period: Middle June until middle of November in middle European climate.

### Reproductive Organs

Stamens:

Number.—35.

Anther.—Length: 0.1 cm. Color: Brown.

Filament.—Length: 1 cm. Color: Yellow. Pollen color: Brown.

Pistils.—

Style.—White-green in color.

Stigma.—Yellow in color.

I claim:

1. A new and distinct variety of rose plant substantially as shown and described.
